(19) United States
(12) Patent Application Publication
(10) Pub. No.: US 2015/0238590 A1
(43) Pub. Date: Aug. 27, 2015

(54) USE OF THE SALMONELLA SPP TYPE III SECRETION PROTEINS AS A PROTECTIVE VACCINATION

(71) Applicant: THE BOARD OF REGENTS FOR OKLAHOMA STATE UNIVERSITY, Stillwater, OK (US)

(72) Inventors: Wendy L. Picking, Stillwater, OK (US); William D. Picking, Stillwater, OK (US)

(73) Assignee: The Board of Regents for Oklahoma State University, Stillwater, OK (US)

(21) Appl. No.: 14/437,535

(22) PCT Filed: Oct. 22, 2013
(86) PCT No.: PCT/US13/66105

§ 371 Date: Apr. 22, 2015

Related U.S. Application Data
(60) Provisional application No. 61/716,911, filed on Oct. 22, 2012.

(51) Int. Cl. A61K 39/112

(52) U.S. Cl. CPC A61K 39/0275; A61K 2039/552

(57) ABSTRACT

Salmonella enteric serotype Typhimurium is a causative agent for gastroenteritis. It is the leading cause of hospitalization and death caused by a food-borne pathogen in the US. As is the case with many gram- negative pathogens, Salmonella spp. use type III secretion systems (T3SS) to deliver proteins to host cells to induce infections. The T3SS uses a molecular syringe and needle, the type III secretion apparatus (T3SA), as a conduit for the transport of the T3SS proteins from the bacteria to the host. Because proteins associated with the tip of the T3SA are extracellular, they are potential protective subunit vaccine candidates against Salmonella. These pathogens also have a T3SS that is involved with intracellular escape from the vacuole with an associated T3SA. Within this invention, we begin to show proof of concept that these proteins are protective.

### CROSS REFERENCE TO RELATED APPLICATION

This application claims the benefit of U.S. Provisional Application No. 61/716,911 filed Oct. 22, 2012, herein incorporated by reference in its entirety for all purposes.

### FIELD OF THE INVENTION

The invention generally relates to protecting against Salmonella-type pathogens and, more particularly, to compositions and methods for immunizing against infection by typhoidal and non-typhoidal Salmonella serovars.

### BACKGROUND

Salmonella is a genus of over 2000 serovars and includes organisms that cause a wide range of human and animal diseases. For example, Salmonella enterica serovars Typhi and Paratyphi A and B cause enteric (“typhoid”) fever. Salmonella enterica serovars Typhimurium and Enteritidis are known as the non-typhoidal Salmonella (NTS) and cause salmonellosis—a gastroenteritis which is usually a self-limiting illness in healthy individuals.

As is the case with many gram-negative pathogens, Salmonella spp. use type III secretion systems (T3SSs) as virulence factors to deliver proteins into host cells and to subsequently cause/induce infection. The T3SS is a molecular “syringe and needle” apparatus, also known as a “type III secretion apparatus” (T3SA) which promotes uptake of the bacterium by the host cell, and then adaptation of the intracellular environment of the host cell to allow a productive infection. Salmonella has two functionally distinct T3SS's which are encoded by Salmonella “pathogenicity islands” 1 and 2 (SPI-1 and -2). The SPI-1 T3SS is central to the ability of Salmonella to invade nonphagocytic cells via the injection, from the bacteria and into the cell by way of the T3SA conduit, effector proteins which trigger extensive actin rearrangements on the surface of host cells. While this allows ingress of the pathogen into the host cell, a second T3SS island, SPI-2, is essential for bacterial replication/proliferation inside host cells. Upon intracellular activation of SPI-2, the bacteria proliferate within membrane-bound vacuoles of phagocytic eukaryotic cells (Salmonella-containing vacuoles, SCVs), with macrophages being the main cell type supporting bacterial growth in vivo. Bacterial effector proteins are translocated across the vacuolar membrane via the SPI-2 T3SS apparatus and into the host endomembrane system and cytoplasm, causing systemic disease.

The Salmonella NTS serotypes are a primary cause of foodborne illnesses worldwide. In the U.S. NTS are a leading cause of hospitalization and death due to foodborne illnesses, with Salmonella enterica serovar Typhimurium being the most frequent cause. 95% of the total cases of NTS are caused by contaminated food. Unfortunately, absolute protection from infection by enhanced agricultural surveillance is not feasible. Vaccines against these pathogens could provide a major weapon in controlling this disease. However, although some progress has been made in recent years, vaccines against Salmonella spp. have not proven to be broadly protective, and almost all are entirely directed only to the typhoid causing serovars. A Salmonella serotype-independent subunit vaccine that could target both typhoid and NTS serovars would be of tremendous public health value.

Heretofore, as is well known in the immunization arts, there has been a need for an invention to address and solve the disadvantages of prior art methods by providing a broad-based serotype-independent Salmonella vaccine. Accordingly, it should now be recognized, as was recognized by the present inventors, that there exists, and has existed for some time, a very real need for a system and method that would address and solve the above-described problems.

Before proceeding to a description of the present invention, however, it should be noted and remembered that the description of the invention which follows, together with the accompanying drawings, should not be construed as limiting the invention to the examples (or preferred embodiments) shown and described. This is so because those skilled in the art to which the invention pertains will be able to devise other forms of the invention within the ambit of the appended claims.

### SUMMARY OF THE INVENTION

Proteins associated with the tip of the T3SA in both SPI-1 and SPI-2 are extracellular, and thus are excellent candidates for the development of broadly protective serotype-independent subunit vaccines against Salmonella. Herein, the successful use of extracellular SPI-1 and SPI-2 proteins to immunize mammals against the effects of Salmonella infection is shown. Accordingly, compositions (e.g. immunogenic compositions) comprising one or more of the SPI-1 and SPI-2 proteins, or immunogenic fragments thereof, are provided, as are methods of using the compositions to elicit an immune response in and/or to vaccinate a mammal. Advantageously, in some aspects the methods and compositions provide broad serovar-independent protection against infection by both typhoid and NTS Salmonella serovars.

In one aspect, the invention provides methods of eliciting an immune response against at least one Salmonella serovar in a subject in need thereof. The methods comprise the steps of administering to the subject a composition comprising i) at least one Salmonella pathogenicity island 1 (SPI-1) and/or Salmonella pathogenicity island 2 (SPI-2) extracellular protein; and ii) a physiologically acceptable carrier; wherein said composition is administered in an amount so as to elicit an immune response to the at least one Salmonella serovar in said subject. In some aspects, the composition further comprises an adjuvant. In other aspects, the composition comprises an extracellular protein selected from the group consisting of: SipD, SipB, SseB and SseC. For example, the composition may comprise SipD and SipB; and the composition may further comprises SseB. In some aspects, the Salmonella serovar is Salmonella enterica serovar. In further aspects, the at least one Salmonella enterica serovar may be: typhoid serovar Typhi, typhoid serovar Paratyphi A, typhoid serovar Paratyphi B, non-typhoidal serovar Typhimurium and non-typhoidal serovar Enteritidis. In additional aspects, the subject is selected from a human and an agricultural animal, with exemplary agricultural animals including cattle, poultry, swine, horses, sheep and goats.

In other aspects, the invention provides immunogenic compositions comprising i) at least one Salmonella pathogenicity island 1 (SPI-1) and/or Salmonella pathogenicity island 2 (SPI-2) extracellular protein; and ii) a physiologically acceptable carrier. In some aspects, the immunogenic composition further comprises an adjuvant. In further aspects, the at least one SPI-1 and/or SPI-2 extracellular protein is SipD, SipB, SseB or SseC. In some aspects, the at least one SPI-1 and/or SPI-2 extracellular proteins in the immunogenic compositions include SipD and SipB. In other aspects, the immunogenic compositions further comprise SseB.

In other aspects of the invention, what is provided is methods of treating or preventing Salmonella infection by one or both of a typhoid Salmonella serovar and a non-typhoid Salmonella serovar in a subject in need thereof. The methods comprise administering to the subject an amount of a composition comprising i) at least one Salmonella pathogenicity island 1 (SPI-1) and/or Salmonella pathogenicity island 2 (SPI-2) extracellular protein; and ii) a physiologically acceptable carrier. In some aspects, the immunogenic composition further comprises an adjuvant. In further aspects, the at least one SPI-1 and/or SPI-2 extracellular protein is SipD, SipB, SseB or SseC. In some aspects, the at least one SPI-1 and/or SPI-2 extracellular proteins in the immunogenic compositions include SipD and SipB. In other aspects, the immunogenic compositions further comprise SseB. The amounts that are administered are sufficient to treat or prevent said Salmonella infection in said subject.

In yet other aspects, the invention provides methods of lessening the severity of symptoms of Salmonella infection in a subject in need thereof, comprising administering to the subject an amount of a composition comprising i) at least one Salmonella pathogenicity island 1 (SPI-1) and/or Salmonella pathogenicity island 2 (SPI-2) extracellular protein; and ii) a physiologically acceptable carrier. In some aspects, the immunogenic composition further comprises an adjuvant. In further aspects, the at least one SPI-1 and/or SPI-2 extracellular protein is SipD, SipB, SseB or SseC. In some aspects, the at least one SPI-1 and/or SPI-2 extracellular proteins in the immunogenic compositions include SipD and SipB. In other aspects, the immunogenic compositions further comprise SseB. The amounts that are administered are sufficient to lessen the severity of said symptoms in said subject.

In additional aspects, the invention provides methods of decreasing fecal shedding of Salmonella from a subject who is or is likely to be infected with Salmonella, comprising administering to the subject an amount of a composition comprising i) at least one Salmonella pathogenicity island 1 (SPI-1) and/or Salmonella pathogenicity island 2 (SPI-2) extracellular protein; and ii) a physiologically acceptable carrier. In some aspects, the immunogenic composition further comprises an adjuvant. In further aspects, the at least one SPI-1 and/or SPI-2 extracellular protein is SipD, SipB, SseB or SseC. In some aspects, the at least one SPI-1 and/or SPI-2 extracellular proteins in the immunogenic compositions include SipD and SipB. In other aspects, the immunogenic compositions further comprise SseB. The amount that is administered is sufficient to lessen the severity of said symptoms in said subject.

The foregoing has outlined in broad terms the more important features of the invention disclosed herein so that the detailed description that follows may be more clearly understood, and so that the contribution of the instant inventors to the art may be better appreciated. The instant invention is not limited in its application to the details of the construction and to the arrangements of the components set forth in the following description or illustrated in the drawings. Rather the invention is capable of other embodiments and of being practiced and carried out in various other ways not specifically enumerated herein. Additionally, the disclosure that follows is intended to apply to all alternatives, modifications and equivalents as may be included within the spirit and the scope of the invention as defined by the appended claims. Further, it should be understood that the phraseology and terminology employed herein are for the purpose of description and should not be regarded as limiting, unless the specification specifically so limits the invention.

### BRIEF DESCRIPTION OF THE DRAWINGS

Other objects and advantages of the invention will become apparent upon reading the following detailed description and upon reference to the drawings in which:

FIG. 1. Schematic illustration of the mouse testing protocol for Example 1.

FIG. 2. IgG antibody titers from mice immunized with T3SS proteins at day 28. Each bar represents data from pooled samples (N=10).

FIG. 3. Survival after challenge. Balb-c mice (N=5 per group) were vaccinated twice with attenuated Salmonella vaccine strain Aro, or 3 times with of a composition comprising SipB, SipD and SseB protein (10 μg of each) with or without adjuvant dmLT. Mice were challenged vias orogastric challenge with 1×10⁶ CFU of Salmonella strain SL1344. Survival was monitored for 14 days after challenge.

FIG. 4 contains a schematic illustration of the mouse testing protocol for Example 2.

FIG. 5A-F. A and B, number of SipB specific ASCs in spleens of immunized mice at days 42 and 56, respectively; C and D, number of SipD specific ASCs at days 42 and 56, respectively; E and F, number of SseB specific ASCs at days 42 and 56, respectively.

FIG. 6A-C. IgG titers in immunized mice at day 56. A, SipB specific IgG; B, SipD specific IgG; C, SseB specific IgG.

FIG. 7. Stool IgA titers in immunized mice at day 56.

FIGS. 8A and B. Protection efficacy in immunized mice after challenge (at day 56) with A, S. enterica Typhi or B, S. enterica Typhimurium.

FIG. 9. Schematic illustration of the calf testing protocol for Example 3.

FIGS. 10A and B. Antibody titers of calves immunized as described in Example 3, on day 56 post-immunization. A, serum IgG; B, saliva IgA,

FIGS. 11A and B. Bacterial shedding in response to challenge with A, S. enterica Newport or B, S. enterica Typhimurium in calves on day 56 post-immunization.

FIG. 12A-F. Sequences of proteins of interest and nucleic acid sequence encoding them. A, amino acid sequence of SipB (SEQ ID NO: 1); B, nucleic acid sequence encoding SipB (SEQ ID NO: 2); C, amino acid sequence of SipD (SEQ ID NO: 3); D, nucleic acid sequence encoding SipD (SEQ ID NO: 4); E, amino acid sequence of SseB (SEQ ID NO: 5); F, nucleic acid sequence encoding SseB (SEQ ID NO: 6).

FIGS. 13A and B. A, Amino acid and B, encoding nucleic acid sequences of SipB-SipD chimera (SEQ ID NOS: 7 and 8, respectively).

### DETAILED DESCRIPTION

While this invention is susceptible of embodiment in many different forms, there is shown in the drawings, and will herein be described hereinafter in detail, some specific embodiments of the instant invention. It should be understood, however, that the present disclosure is to be considered an exemplification of the principles of the invention and is not intended to limit the invention to the specific embodiments or algorithms so described.

The surface-exposed proteins at the tip of the two type III secretion (TTS) apparatus needles of Salmonella (both SPI-1 and SPI-2) have been used for the development of vaccines protective against all S. enterica subspecies. TTS apparatus needle tip proteins from Salmonella which have been used in this manner include but are not limited to: the initial tip protein SipD and the first ‘translocator’ protein SipB from Salmonella pathogenicity island-1 (SPI-1). Without being bound by theory, it is believed that disruption of the function of SPI-1 (e.g. by antibody binding to one or more of these proteins) prevents Salmonella protein delivery to host cells and thus blocks bacterial effects on the host cell. In addition, after entry into the cell, the proteins from SPI-2 are expressed and are surface exposed, and these proteins, which include but are not limited to: SseB and SseC, also provide vaccine targets. Without being bound by theory, it is believed that disruption of the SPI-2 apparatus (e.g. by antibody binding to one or more of these proteins) prevents successful replication of the bacteria within the host, and also blocks spread of the bacteria within the host, preventing systemic infection.

Accordingly, proteins which make up SPI-1 and/or the SPI-2 of Salmonella, or antigenic portions thereof, are used to elicit an immune response in subjects to whom they are administered. By “elicit an immune response” we mean that the subject mounts one or both of an innate and/or an adaptive immune reaction against antigenic determinants of the proteins or antigenic portions thereof that are administered. In particular, the adaptive immune reaction entails production of e.g. B and T cell lymphocytes and antibodies specific for binding and forming complexes with the antigenic determinants. In some embodiments, the proteins and/or antigenic fragments thereof elicit a protective immune response in the subject, i.e. administration of one or more of the proteins and/or antigenic portions thereof results in an immune response that is protective against later challenge by the disease causing organism itself, either preventing infection altogether, or lessening the impact of infection by decreasing disease symptoms that would otherwise occur, had the subject not been vaccinated as described herein.

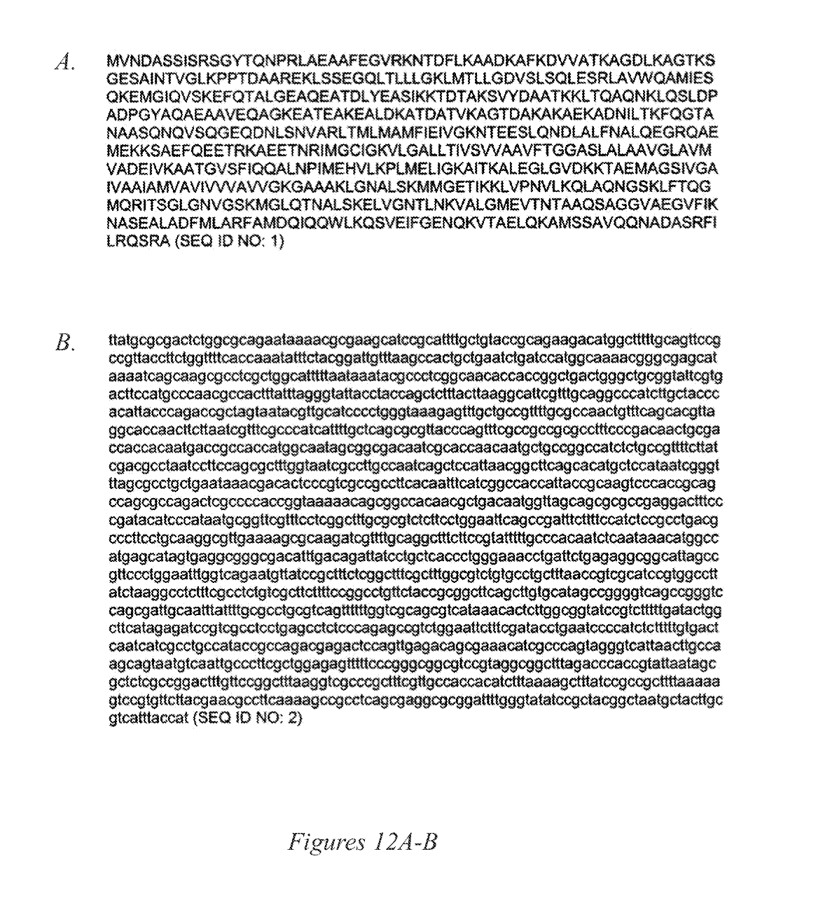

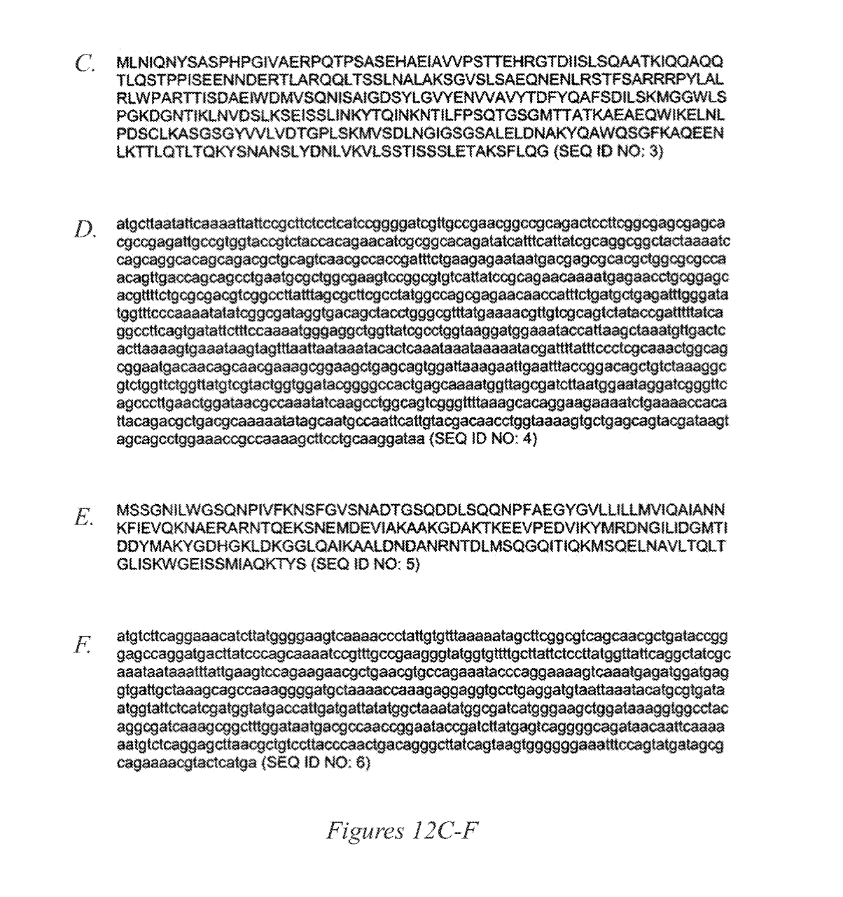

Exemplary proteins which may be used in the practice of the invention include but are not limited to: SipD, SipB, SseB and SseC, which may be administered alone or in various forms and combinations as described herein. Exemplary full length sequences of SipD, SipB, and SseB and nucleic acids that encode them are provided in FIG. 12A-F (SEQ ID NOS: 1-6). The phrase(s) “SipD, SipB, SseB and SseC protein(s)” as used herein refer to both the full length proteins as depicted in the figures, and also to antigenic regions (portions, fragments, etc.) thereof. By an “antigenic region” we mean a foreshortened or truncated version or fowl of the protein which elicits the same level or a comparable level of protection as does the full length protein from which it is taken, a comparable level being in the range of at least about 50, 55, 60, 65, 70, 75, 80, 85, 90, 95, or even 100% (or more) of the protective activity of the parent, full-length molecule. An antigenic region may be a shorter protein (e.g. containing more than about 250 amino acids), a polypeptide (e.g. containing less than about 250 amino acids but more than about 100 amino acids), or a peptide (e.g. containing less than about 100 amino acids). Those of skill in the art will recognize that the “definitions” of protein, polypeptide and peptide may vary within the art, and may overlap in meaning, and are often used interchangeably, as may be the cases herein.

The proteins, polypeptides/peptides which may be used in the practice of the invention include SipD, SipB, SseB and SseC, and variants or derivatives thereof which have at least about 50, 55, 60, 65, 70, 75, 80, 85, or 90% identity to the sequences presented herein, or to sections of those sequences which correspond to antigenic regions. Typically, the level of identity is at least about 92, 93, 94, 95, 96, 97, 98 or 99%. Those of skill in the art are familiar with methods and software programs for sequence comparison to determine identity.

The invention also encompasses the use of nucleic acids encoding the proteins/polypeptides/peptides described herein, i.e. nucleic acid vaccines are also contemplated, as are vectors for producing the proteins. Exemplary nucleic acid sequences encoding e.g. SipD, SipB, and SseB are presented in FIG. 12 B, D and F (SEQ ID NOS: 2, 4 and 6, respectively). However, those of skill in the art will appreciate that the genetic code is redundant and that many sequences may encode a given amino acid sequence. All nucleic acid sequences (e.g. DNA, RNA, cDNA, DNA/RNA hybrids, etc.) which successfully encode and express the proteins/polypeptides/peptides described herein may be used in the practice of the invention, so long as administration results in elicitation of an immune response (e.g. a protective immune response) as described herein.

Also encompassed are vectors which contain such nucleic acid sequences (e.g. plasmids, cosmids, various expression vectors, etc.), and host cells such as bacteria, insect cells, etc., which comprise the nucleic acid sequences and/or the vectors. Such vectors typically include one or more expressible gene sequences encoding one or more or the proteins/polypeptides/peptides of interest, operably linked to at least one transcription element (e.g. a promoter) that drives expression of the gene. The vectors may be used for production of the proteins/fragments thereof, or may be used as a vaccinating agent, i.e. the invention also encompasses nucleic acid vaccines. Those of skill in the art are aware of the many protocols for preparing and administering nucleic acid vaccines, such as those described in issued U.S. Pat. Nos. 7,927,870 and 7,094,410, the complete contents of which are hereby incorporated by referenced in entirety.

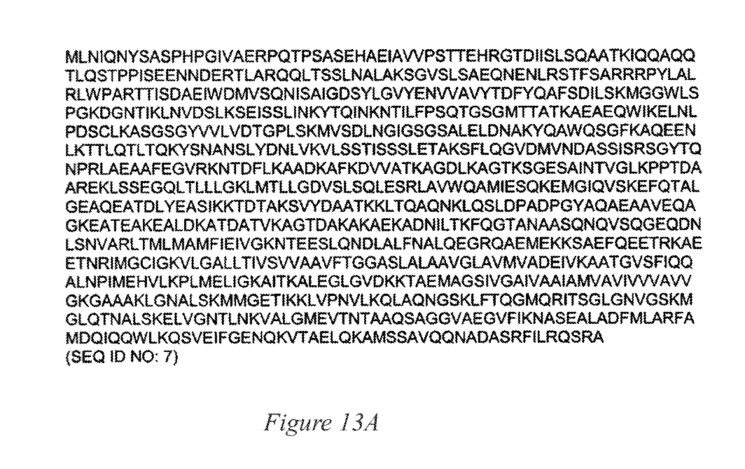

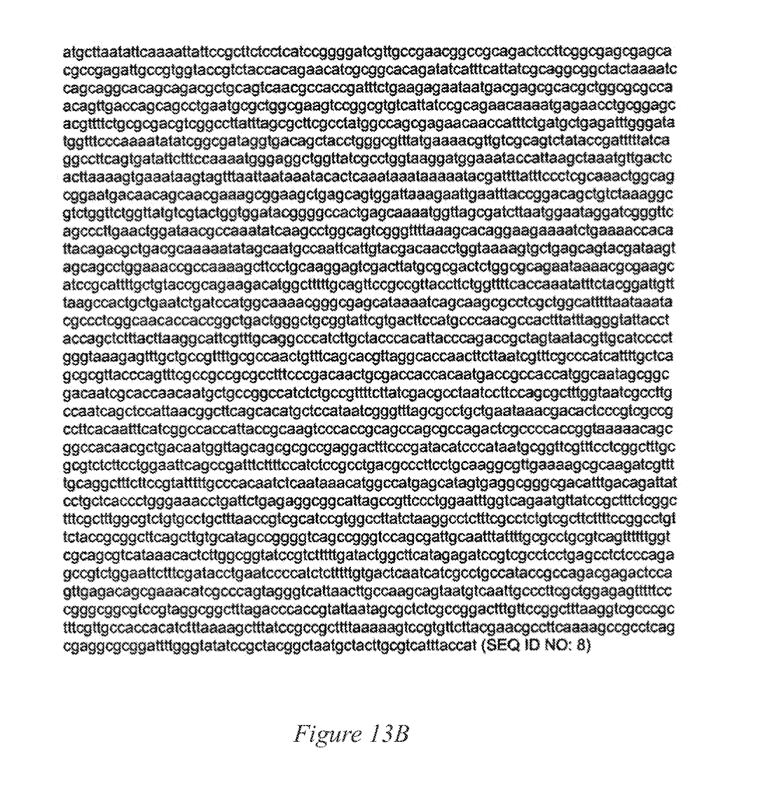

In some embodiments of the invention, the proteins, polypeptides or peptides that are used in the vaccine are chimeric or fusion proteins. By a “chimeric protein” we mean a protein that is translated from a single, contiguous nucleic acid molecule, and which comprises sequences from at least two different proteins or antigenic regions thereof. For example, a chimera of the invention may include two or more of SipD, SipB, SseB and SseC, or antigenic regions of two or more of these. Typically, the individual sequences are joined via a linker or spacer sequence of e.g. from about 2 to about 20 amino acids, usually from about 2 to about 10 amino acids. The amino acids in linking sequences are typically uncharged and the linker sequence usually does not exhibit secondary or tertiary structure, but does allow the fused protein/peptide segments to adopt functional secondary, tertiary, etc. conformations. One such exemplary chimera includes SipB and SipD. The amino acid sequence of this chimera is shown in FIG. 13A (SEQ ID NO: 7). The chimera may be encoded by any suitable nucleic acid sequence, e.g. the exemplary nucleic acid sequence depicted in FIG. 13B (SEQ ID NO: 8).

The present invention provides compositions for use in eliciting an immune response and/or vaccinating an individual against Salmonella infection, and/or against disease symptoms caused by Salmonella infection. The compositions include one or more substantially purified proteins, polypeptides or antigenic regions thereof as described herein, or substantially purified nucleic acid sequences (e.g. DNA cDNA, RNA, etc.) encoding such proteins, polypeptides or antigenic regions thereof, and a pharmacologically suitable/compatible carrier. By “substantially purified” we mean that the molecule is largely free of other organic molecules, cellular debris, solvents, etc. when tested using standard techniques known to those of skill in the art (e.g. gel electrophoresis, column chromatography, sequencing, mass spectroscopy, etc.). For example, the molecule is generally at least about 50, 55, 60, 65, 70, or 75% pure by wt/%, and preferably is at least about 80, 85, 90, 95% or more pure (e.g. 96, 97, 98, 99 or even 100% pure). The preparation of proteins, polypeptides, and peptides as described herein is well-known to those in the art, and includes, for example, recombinant preparation; isolation from a natural source; chemical synthesis; etc. The purification of proteinaceous materials is also known. However, specific exemplary methods for preparing the vaccinating agents utilized in the practice of the invention are described in detail in the Examples section below.

The preparation of compositions for use as vaccines is known to those of skill in the art. Typically, such compositions are prepared either as liquid solutions or suspensions, however solid forms such as tablets, pills, powders and the like are also contemplated. Solid forms suitable for solution in, or suspension in, liquids prior to administration may also be prepared (e.g. lyophilized, freeze-dried forms, etc.). The preparation may also be emulsified. The active ingredients may be mixed with excipients which are pharmaceutically acceptable and compatible with the active ingredients. Suitable excipients are, for example, water, saline, dextrose, glycerol, ethanol and the like, or combinations thereof In addition, the composition may contain minor amounts of auxiliary substances such as wetting or emulsifying agents, pH buffering agents, and the like. If it is desired to administer an oral form of the composition, various thickeners, flavorings, diluents, emulsifiers, dispersing aids or binders and the like may be added. The composition of the present invention may contain any such additional ingredients so as to provide the composition in a form suitable for administration. The final amount of protein, polypeptide or peptide (or encoding nucleic acids) in the formulations may vary. However, in general, the amount of vaccinating/immunizing agent in the formulations will be from about 1 to about 99%.

In addition, the composition may contain adjuvants, many of which are known in the art. For example, adjuvants suitable for use in the invention include but are not limited to: bacterial or microbial derivatives such as non-toxic derivatives of enterobacterial lipopolysaccharide (LPS), Lipid A derivatives, immunostimulatory oligonucleotides and ADP-ribosylating toxins and detoxified derivatives thereof. Non-toxic derivatives of LPS include monophosphoryl lipid A (MPL) and 3-O-deacylated MPL (3dMPL). 3dMPL is a mixture of 3 de-O-acylated monophosphoryl lipid A with 4, 5 or 6 acylated chains. A preferred “small particle” form of 3 De-O-acylated monophosphoryl lipid A. Other non-toxic LPS derivatives include monophosphoryl lipid A mimics, such as aminoalkyl glucosaminide phosphate derivatives e.g. RC-529. Lipid A derivatives include derivatives of lipid A from Escherichia coli such as OM-174. Immunostimulatory oligonucleotides suitable for use as adjuvants in the invention include nucleotide sequences containing a CpG motif (a dinucleotide sequence containing an unmethylated cytosine linked by a phosphate bond to a guanosine). Double-stranded RNAs and oligonucleotides containing palindromic or poly(dG) sequences have also been shown to be immunostimulatory. The CpG's can include nucleotide modifications/analogs such as phosphorothioate modifications and can be double-stranded or single-stranded. e.g. replacement of guanosine with 2’-deoxy-7-deazaguanosine. The CpG sequence may include, for example, the motif GTCGTT or TTCGTT. The CpG sequence may be specific for inducing a Thl immune response, such as a CpG-A ODN, or it may be more specific for inducing a B cell response, such a CpG-B ODN. CpG-A and CpG-B ODNs. Preferably, the CpG is a CpG-A ODN. Preferably, the CpG oligonucleotide is constructed so that the 5′ end is accessible for receptor recognition. Optionally, two CpG oligonucleotide sequences may be attached at their 3′ ends to form “immunomers”.

Bacterial ADP-ribosylating toxins and detoxified derivatives thereof may be used as adjuvants in the invention. Preferably, the protein is derived from E. coli (e.g. E. coli heat labile enterotoxin “LT”), cholera (“CT”), or pertussis (“PT”). The use of detoxified ADP-ribosylating toxins as mucosal adjuvants and as parenteral adjuvants is known. The toxin or toxoid is preferably in the form of a holotoxin, comprising both A and B subunits. Preferably, the A subunit contains a detoxifying mutation; preferably the B subunit is not mutated. Preferably, the adjuvant is a detoxified LT mutant such as LT-K63, LT-R72, and LT-G192. The use of ADP-ribosylating toxins and detoxified derivatives thereof, particularly LT-K63 and LT-R72, is known. Such adjuvants are described, for example, in issued U.S. Pat. No. 8,039,007 (the complete contents of which is hereby incorporated by reference in entirety). Various interleukins may also be used as adjuvants to increase the immune response in a subject. In preferred embodiments, the adjuvant is a mucosal adjuvant such as, for example, the double mutant heat-labile toxin (dmLT) from enterotoxigenic E. coli.

The vaccine compositions (preparations) of the present invention may be administered by any of the many suitable means which are well known to those of skill in the art, including but not limited to by injection, inhalation, orally, intranasally, topically, inclusion in a food product, etc. In preferred embodiments, the mode of administration is intradermal or intramuscular. In addition, the compositions may be administered in conjunction with or in a composition which contains other antigens of interest. In other words, they may be administered as a component of a multivalent vaccine which also contains antigens against other related or non-related infectious diseases, e.g. childhood diseases, such as polio, whooping cough, tetanus, diphtheria, etc.

Recipients of the vaccine of the invention are generally mammals, frequently humans, but this is not always the case. While in some aspects, the vaccine is used to prevent illness in humans, in other aspects, the vaccine is used to prevent illness and/or to block the carrier states in agriculturally important animals, i.e. veterinary applications are also contemplated. Animals which could benefit from receiving the vaccine/immune eliciting compositions of the invention include but are not limited to: cattle, poultry, swine, horses, sheep and goats. The vaccine is generally delivered intranasally or subcutaneously or intramuscularly, and may be delivered in combination with other agents such as other vaccinogens.

For humans, the vaccines may be administered alone or together with so-called “child hood” vaccines. Thus, the recipients are preferably human children who may be infants (e.g. up to about 1 hear of age), toddlers (e.g. up to about 2 years of age), or older, and administration may be carried out as a series of initial inoculations followed by booster doses at suitable intervals, e.g. monthly, or every 6-months, or yearly, etc. as necessary to provide protection. Thereafter, or in the case of adults who have not previously been vaccinated, the vaccines may be administered as necessary to result in a protective immune response, and subjects may be re-boosted e.g. about every 10 years throughout adult life. Of special interest is vaccination of individuals with compromised immune systems, e.g. the elderly, those receiving cancer or other immune weakening therapy, those afflicted with HIV, etc.

Vaccine recipients may have never been exposed to Salmonella, or may have been exposed or suspected of having been exposed but be asymptomatic, or may have actual symptoms of disease, and still benefit from administration of the vaccine. Vaccine administration may prevent disease symptoms entirely, or may lessen or decrease disease symptoms, the latter outcome being less than ideal but still better than experiencing full-blown disease symptoms.

The amount of protein that is administered per dose of vaccine is typically in the range of from about 1 to about 1000 μg/kg, and is usually in the range of from about 0.02 to about 20 μg/kg of body weight of the recipient. Those of skill in the art will recognize that the precise dosage may vary from situation to situation and from patient to patient, depending on e.g. age, gender, overall health, various genetic factors, and other variables known to those of skill in the art. Dosages are typically determined e.g. in the course of animal and/or human clinical trials as conducted by skilled medical personnel, e.g. physicians.

The vaccines and immunogenic compositions of the invention are broad-based vaccines and may be used to provide immune protection against a variety of Salmonella species, including both typhoidal and non-typhoidal species, and all S. enterica subspecies. Examples of these two categories of Salmonella include but are not limited to: for typhoidal Salmonella: Paratyphi A&B; and for non-typhoidal Salmonella: Typhimurium, Enteriditis, Newport, Dublin. Administration of the compositions of the invention results in the production of an immune response, which may be a protective immune response, in subjects who receive the compositions, e.g. by elicitation of antibody production against the administered antigens. Such antibodies are also encompassed by the invention, for example, those generated using laboratory techniques in experimental animals, or using cell culture, or by chemical syntheses, etc. Such antibodies may be polyclonal or monoclonal, and may be specific for the antigens (reacting with no other antigens) or selective for the antigens (reacting more strongly or preferably with the antigens, compared to other antigens). The antibodies may be multivalent. The antibodies may be used for research and/or diagnostic purposes, or alternatively, for treatment, especially of individuals who are infected with Salmonella.

The invention provides methods of vaccinating, or, alternatively, of eliciting an immune response, in a subject in need thereof. The method generally involves identifying a suitable subject, and administering the composition as described herein. The method may also encompass follow-up of administration, e.g. by assessing the production of protective antibodies by the subject, or the presence (or lack thereof) of disease symptoms, etc. The immune response that is elicited may be of any type, i.e. any type of antibody may be produced in response to administration, and cell-mediated immunity may also be elicited.

In addition, the invention provides methods of treating or preventing Salmonella infection by one or both of a typhoid Salmonella serovar and a non-typhoid Salmonella serovar in a subject, methods of lessening the severity of symptoms of Salmonella infection in a subject, and methods of decreasing fecal shedding of Salmonella from a subject who is or is likely to be infected with Salmonella. Each of these methods involves administering to the subject an amount of a composition comprising at least one Salmonella pathogenicity island 1 (SPI-1) and/or Salmonella pathogenicity island 2 (SPI-2) extracellular protein; and a physiologically acceptable carrier. The amount of the composition is administered is sufficient to elicit an immune response to the at least one Salmonella serovar in said subject, thereby of treating or preventing Salmonella infection, lessening the severity of symptoms of Salmonella infection, and/or decreasing fecal shedding of Salmonella.

### EXAMPLES

In the case of Salmonella, an effective vaccine could be used to prevent illness in humans and to block the carrier activity in agriculturally important animals. Although some progress has been made in recent years, available vaccines against Salmonella spp. are not broadly protective and are almost entirely directed at the typhoid causing serovars, even though the non-typhoid serovars are a major public health problem.

Because the SPI-1 and SPI-2 proteins are surface localized prior to the invasion of host cells, they serve as prime targets for subunit vaccines. These four tip proteins are the initial tip protein SipD and the first ‘translocator’ protein SipB from SPI-1, and SseB and SseC from SPI-2, which are expressed and are surface exposed after entry of the bacterium into the cell. We previously demonstrated that the SipD and SipB homologs in Shigella, IpaD and IpaB, respectively, are protective antigens against challenge with serotypically distinct S. flexneri and S. sonnei strains using the mouse pulmonary model (see US patent application 20130149329, the complete contents of which is hereby incorporated by reference in entirety). A similar Salmonella serotype-independent subunit vaccine would be of tremendous public health value. Thus, we have determined the protective efficacy of SipD, SipB and SseB administered with adjuvants against challenge by Salmonella spp.

For Examples 1-3 below, recombinant SipD and SseB were prepared by expression in E. coli using His-tag technology and pET vectors (commercially available from Novagen), and purification was carried out via column chromatography. Within the cytoplasm of Salmonella, SipB forms a complex with its cognant chaperone SicA, and recombinant SipB was prepared with its chaperone by co-expression of His-tagged recombinants in E. coli and co-purification via column chromatography in a method similar to that of Birket et al (Biochemistry [2007] 46:8128-8137). SipB was also produced alone by inducing its release from the chaperone using different detergents.

SipB, SipD, and SseB were then examined as protective antigens by immunizing mice (Examples 1 and 2) and calves (Example 3) intranasally or subcutaneous, followed by challenge, as described below. The results showed that SseB, SipB and SipD are effective in reducing the severity and length of disease caused by Salmonella. These findings impart a significant advancement over the live-attenuated and lipopolysaccaride-based vaccines that are currently being tested, because they provide serotype-independent protection and they can be given to children.

### Example 1

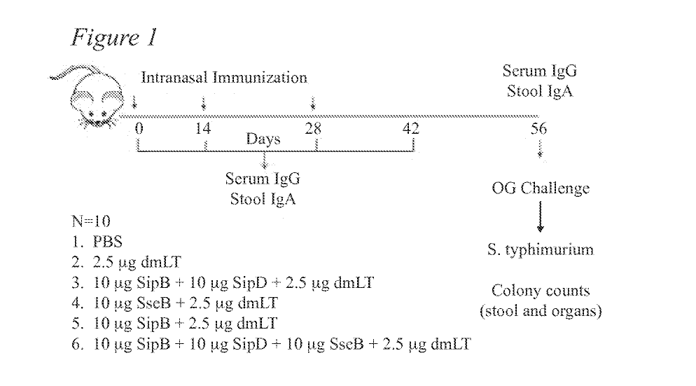

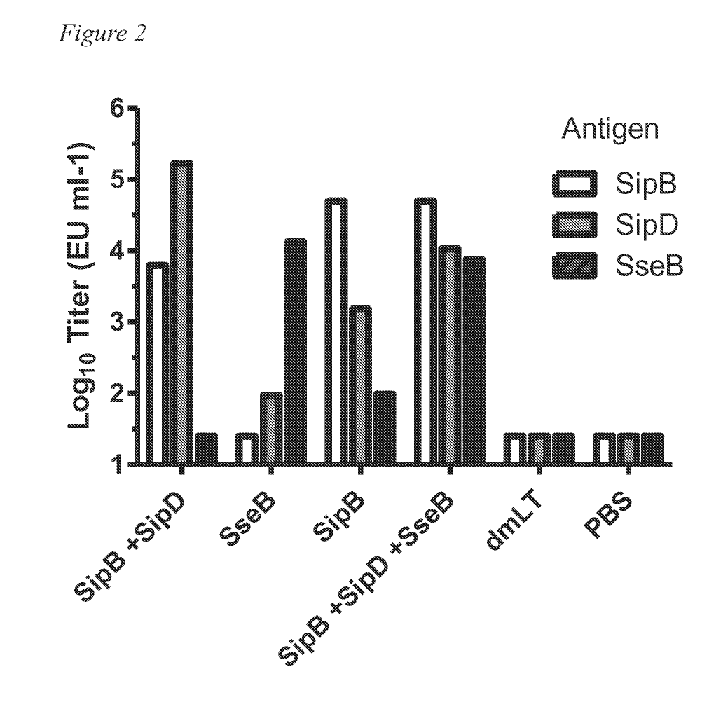

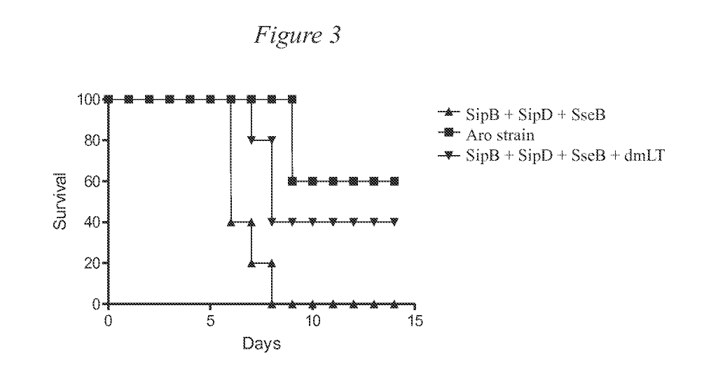

FIG. 1 provides a Schematic illustration of a first mouse testing protocol for Example 1. Briefly, Balb-c mice (N=5 per group) were immunized intranasally 2 times with Aro strain or 3 times (days 0, 14 and 28) with 10 μg each of SipB/SipD/SseB protein with or without dmLT. Serum IgG and stool IgA were monitored throughout. Serum IgG antibody titers at day 28 are shown in FIG. 2. Mice were orogastrically challenged with 10⁸ colony forming units (CFUs) of Salmonella enterica serovar Typhimurium SL1344 after streptomycin treatment on day 56 and survival after challenge was monitored for 14 days after challenge. The results are shown in FIG. 3.

As can be seen, the proteins provide protection against a S. typhimurium challenge at a level approaching the live attenuated vaccine (Aro strain).

### Example 2

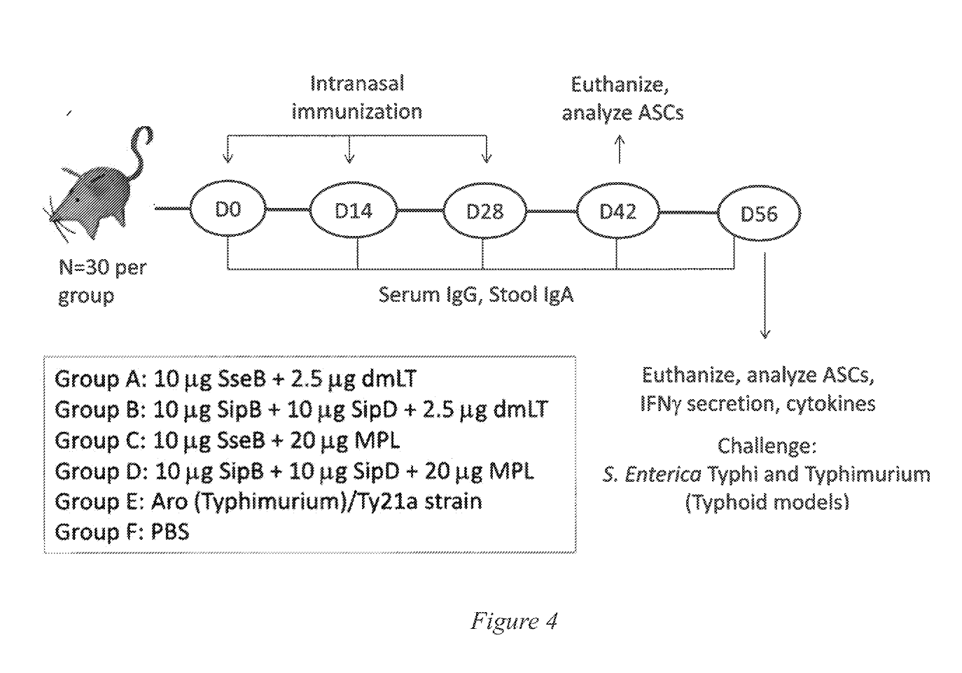

FIG. 4 provides a Schematic illustration of a mouse testing protocol for Example 2. Briefly, Balb-c mice, n=30) were immunized on Days 0, 14 and 28 as follows: Group A: 10 μg SseB+2.5 μg of dmLT (double mutant E. coli heat labile toxin) as adjuvant; Group B: 10 μg SipB+10 μg SipD+2.5 μg dmLT; Group C: 10 μg SseB+20 μg MPL (monophosphooryl Lipid A); Group D: 10 μg SipB+10 μg SipD+20 μg MPL; Group E: Aro (Aro attenuated S. enterica var. Typhimurium vaccine strain, “Typhimurium/Ty21 a strain”); Group F: phosphate buffered saline (PBS, i.e. vehicle). At day 42 post immunization, some mice were euthanized and their spleens were analyzed for antibody secreting cells (ASCs). On day 56, additional mice were euthanized and analyzed for ASCs, IFNγ secretion, and cytokine production, and the remaining mice were challenged with S. enterica typhi or typhimurium (typhoid models).

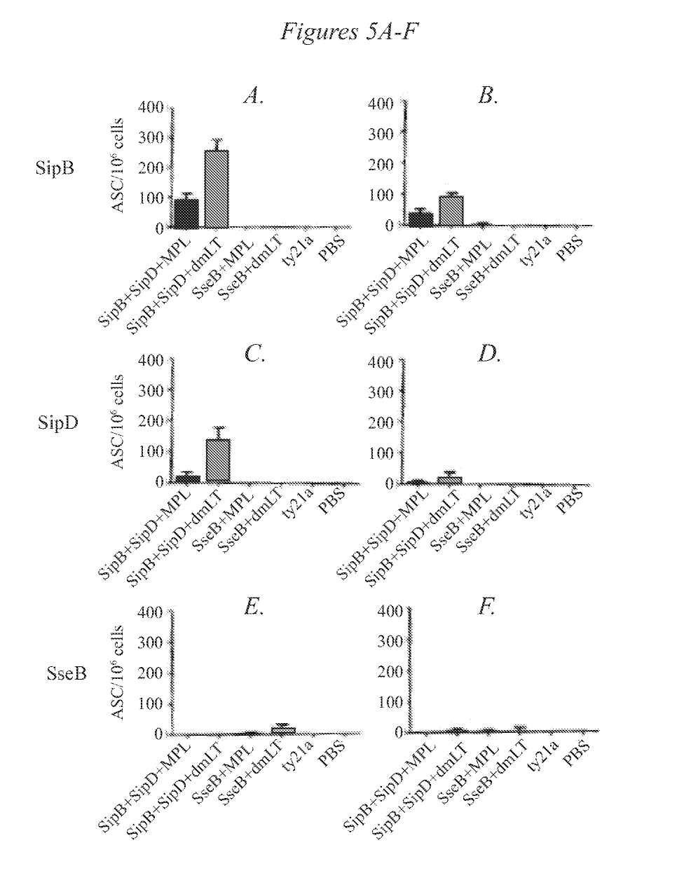

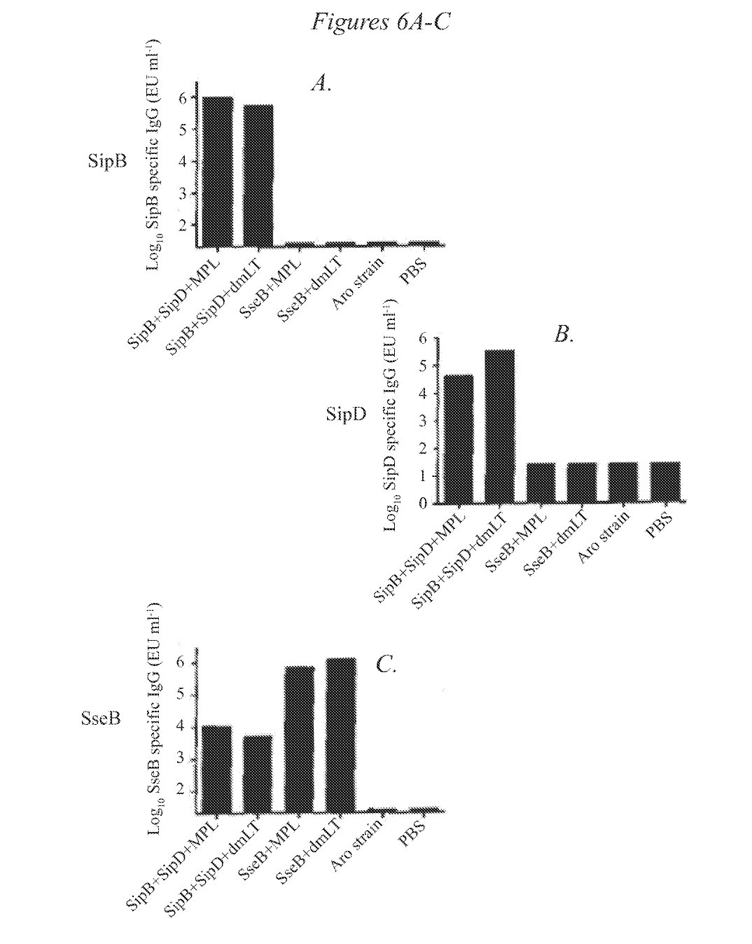

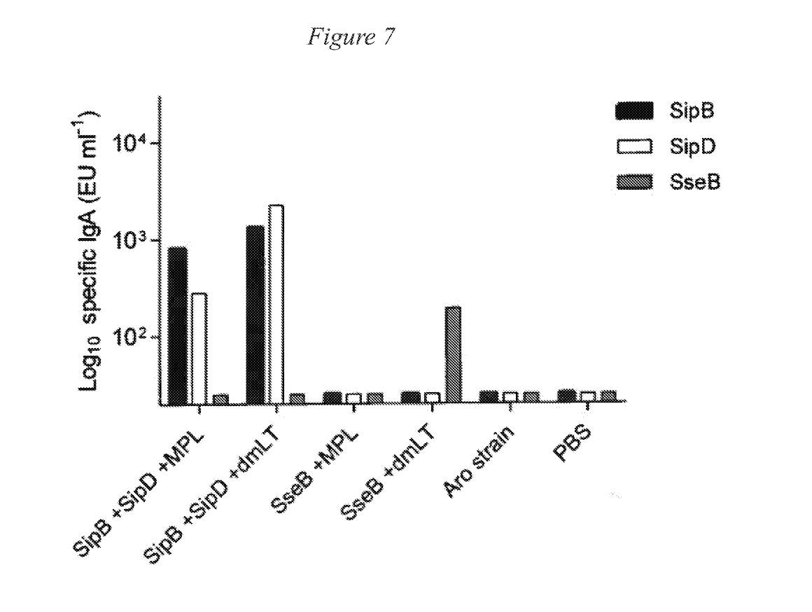

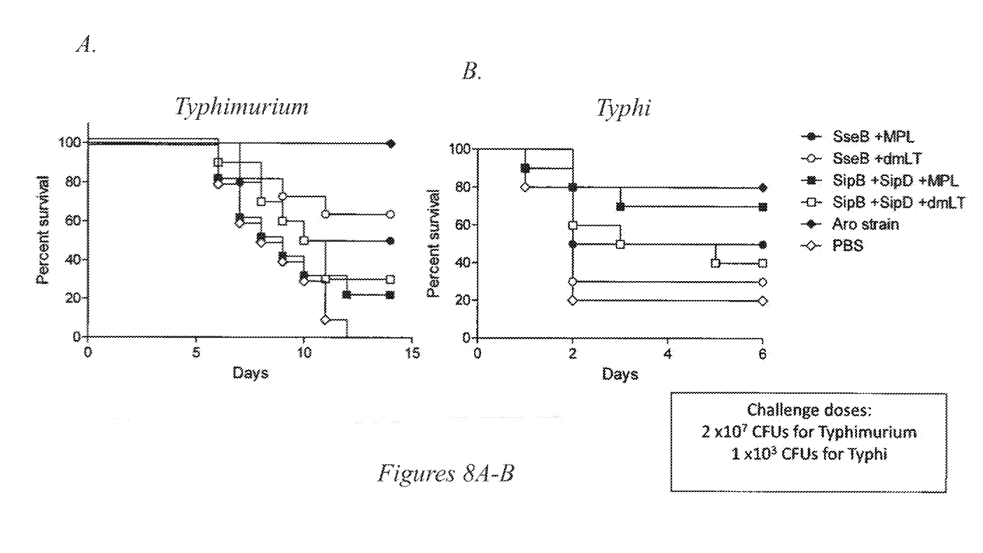

Immunogenicity as shown by spleen ASC results is shown in FIG. 5A-F. FIG. 6A-C shows IgG titers in immunized mice at day 56, FIG. 7 shows stool IgA titers in immunized mice at day 56, and FIG. 8A and FIG. 8B show protection efficacy in immunized mice after challenge (at day 56) with S. enterica typhi (FIG. 8A) or S. enterica typhimurium (FIG. 8B).

As can be seen, the results indicated that SipB, SipD and SseB are munogenic in mice in that both serum and stool antibodies were detected after immunization, as were spleen ASCs specific for SipB and SipD. Interestingly, the challenge experiments showed different outcomes, with the combination of SipB/SipD being protective in Typhi intraperitoneal (IP) mucin challenge model and SseB being protective in the Typhimurium orogastric (OG) challenge model.

### Example 3

### First Calf Testing

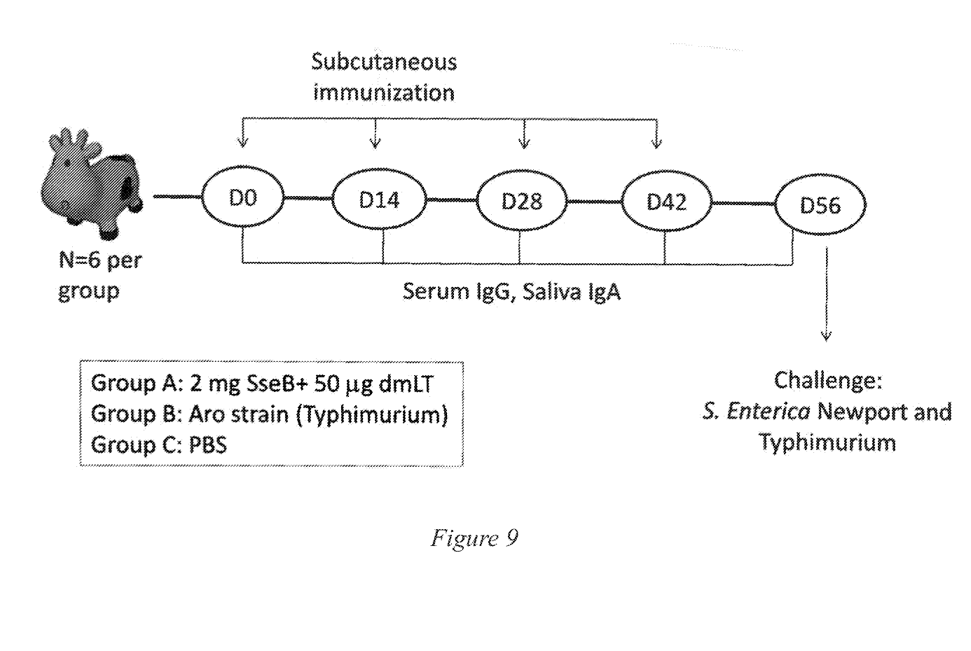

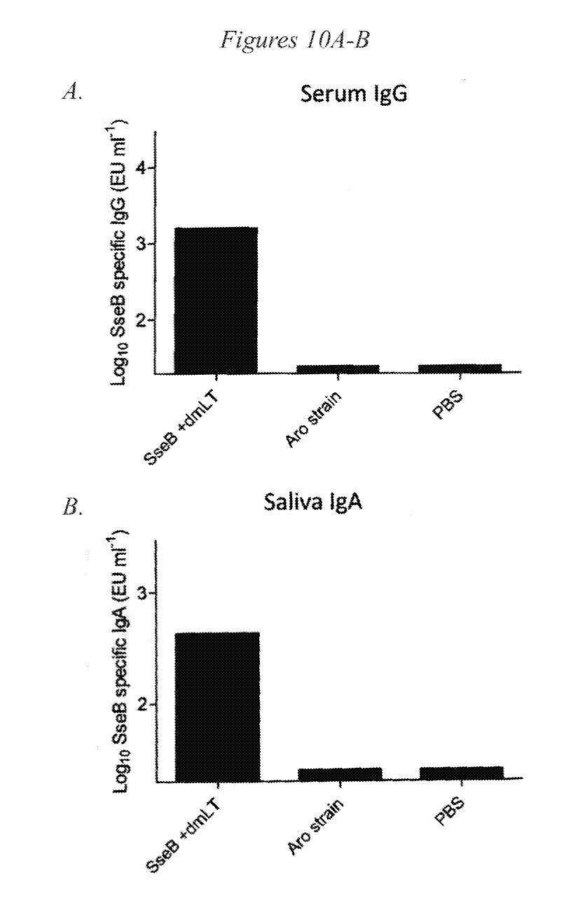

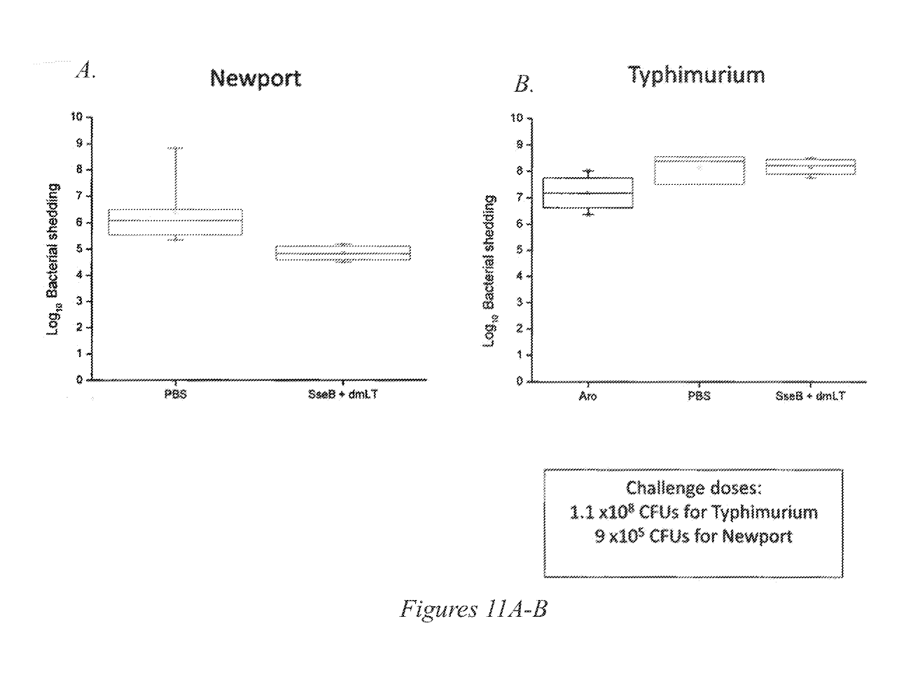

A schematic representation of the experimental protocol is provided in FIG. 9. Calves (21 day old male Holstein or Holstein-Jersey) were immunized subcutaneously at days 0, 14. 28 and 42 as follows: Group A, 2 mg SseB+50 μg of dmLT; Group B, Aro strain (Typhimurium); and Group C, PBS (control). Serum IgG (FIG. 10A) and saliva IgA (FIG. 10B) was measured. Calves were challenged with S. enterica Newport (9×10⁵ CFUs) or Typhimurium (1.1×10⁸ CFUs) on day 56. The amount of bacterial shedding in the two groups of challenged animals is shown in FIGS. 11A and B. As can be seen, the total bacterial shedding over the course of ten days for the group vaccinated with SseB+dmLT was not only lower, but the variation over the course of ten days was less than one log unit which is consistent with no effective increase in shedding over time. This is in contrast to the group vaccinated with PBS which had a higher average shedding and an average over the course of the study that spanned over 2.5 log units. Thus, the SseB+dmLT vaccine reduced disease severity relative to the control group and reduces shedding from the initial time point.

### Example 4

### Preparation of a Chimeric (Fusion) Protein

An SipD-SipB chimeric protein has been developed that allows the protective potential of SipD and SipB to be encompassed in single recombinant protein. This reduces the vaccine composition components to two proteins, the chimeric SipD-SipB and the SipA protein.

The sipD and sipB genes have been genetically fused in a single plasmid and cotransformed into E. coli Tuner (DE3) cells with a plasmid encoding sicA, using the following protocol: combine 1 μg of each plasmid with 30 μl chemically competent cells, heat shock, add one ml LB media, incubate at 37° C. for 1 hr and then plated on LB agar containing 100 μg/ml ampicillin and 30 μg Chloramphenicol. sipD was copied by PCR using primers with NdeI at 5′ end and SalI at 3′ end. sipB was copied by PCR using primers with SalI at 5′ end and XhoI at 3′ end. The PCR fragments were digested with the restriction enzymes and ligated together and then into NdeI/XhoI digested pET15b from Novagen. The ligation product was used to transform NovaBlue E. coli. Transformants were screened for the proper insert and subjected to double stranded sequencing. The gene encoding SicA was copied by PCR from S. Typhimurium SL1344 (accession number J04117). The PCR fragment was digested with the restriction enzymes NdeI and XhoI and ligated into NdeI/XhoI digested pACYCDuet-1 from Novagen. The ligation product was used to transform NovaBlue E. coli. Transformants were screened for the proper insert and subjected to double stranded sequencing. A plasmid containing the correct sequence for the fusion and a plasmid containing the correct sequence for SicA were transformed into Tuner (DE3) E. coli (Novagen). This strain was used to inoculate LB media containing ampicillin and chloramphenicol. The bacteria were grown to an absorbance at 600 of about 0.6 at which time they were induced to over express IpaB/IpgC with Isopropyl-β-D-thio-galactoside (IPTG). The bacteria were grown an additional three hours to allow protein expression to occur. Alternatively, a starter culture of 100 ml was used to inoculate a 5- or 10-liter fermentor vessel containing TB media. After growing overnight (16 hours), the bacteria were induced and allowed to express protein for three hours. The bacteria were collected by centrifugation, resuspended in binding buffer (see below for recipe), and lysed by microfluidization. This suspension was clarified by centrifugation and was loaded onto a nickel charged immobilized metal affinity column (IMAC). The column (5 ml) was washed with 10 bed volumes each of binding and wash buffers and then subjected to a gradient of 0% to 40% elution buffer. Peak fractions were collected, the buffer exchanged into 1M ammonium sulfate in 50 mM sodium phosphate pH 7.0 and loaded onto a Butyl Sepharose High Performance column (5 ml) with a linear gradient from 1M ammonium sulfate in 50 mM sodium phosphate pH 7.0 to 50 mM sodium phosphate. For preparation of His-tag fusion, the IMAC-bound protein complex was incubated overnight in the presence of 1% OPOE. The chaperone was removed in the flow through and subsequent wash steps. His-tag fusion was eluted in the presence of OPOE to maintain the protein in a soluble form. All proteins were concentrated by ultrafiltration and dialyzed into PBS pH 7.2. Protein concentrations were determined via absorbance at 280 nm using extinction coefficients based on the amino acid composition of each protein.

Buffers that were utilized are listed below:

### 4× Charge Buffer: 500 ml

| | | |
|---|---|---|
| 200 mM NiSO₄ | 52.56 g | 1X = 50 mM |

### 8× Binding Buffer: 1Liter

| | | | |
|---|---|---|---|
| 40 mM Imidazole | 2.72 | g | 1X = 5 mM |
| 4M NaCl | 237 | g | 500 mM |

| | | |
|---|---|---|
| 160 mM Tris | 19.36 | 20 mM |

Mix together and pH to 7.9 with HCl
8× Wash Buffer: 500 mL

| | | | |
|---|---|---|---|
| 480 mM Imidazole | 16.3 | g | 1X = 60 mM |
| 4M NaCl | 117 | g | 500 mM |

| | | |
|---|---|---|
| 160 mM Tris | 19.68 | 20 mM |

Mix together and pH to 7.9 with HCl
IF USING UREA drop the imidazole to 20 mM . . . 8×=160
4× Elution Buffer: 500 mL

| 4M Imidazole | 136 g | 1X = 1M |
|---|---|---|
| 2M NaCl | 58.44 | .5M |
| 80 mM Tris | 4.84 | 20 mM |

Mix together and pH to 7.9 with HCl
4× Strip Buffer: 500 mL

| 0.4M EDTA | 74.4 g | 1X = 100 mM |
|---|---|---|
| 2MNaCl | 58.44 | 500 mM |
| 80 mM Tris | 4.84 | 20 mM |

Mix together. Add NaOH pellets to get pH to 8.0 so that EDTA will dissolve. Then adjust pH to 7.9.

A chimeric protein containing both SipD and SipB proteins was produced (FIG. 13; SEQ ID NO: 7), together with SipA protein. This method allows a cGMP facility to produce these proteins with one fermentor run, significantly reducing the protein purification costs.

The chimeric protein is administered to a subject as described herein and, as a result, a protective immune response is elicited in the subject.

The term “at least” followed by a number is used herein to denote the start of a range beginning with that number (which may be a ranger having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%.

When, in this specification, a range is given as “(a first number) to (a second number)” or “(a first number)-(a second number)”, this means a range whose lower limit is the first number and whose upper limit is the second number. For example, 25 to 100 should be interpreted to mean a range whose lower limit is 25 and whose upper limit is 100. Additionally, it should be noted that where a range is given, every possible subrange or interval within that range is also specifically intended unless the context indicates to the contrary. For example, if the specification indicates a range of 25 to 100 such range also is intended to include subranges such as 26 to 100, 27 to 100, etc., 25 to 99, 25 to 98, etc., as well as any other possible combination of lower and upper values within the stated range, e.g., 33-47, 60-97, 41-45, 28-96, etc. Note that integer range values have been used in this paragraph for purposes of illustration only and decimal ranges (e.g., 46.7-91.3) should also be understood to be intended as a possibility unless specifically excluded.

It should be noted that where reference is made herein to a method comprising two or more defined steps, the defined steps can be carried out in any order or simultaneously (except where context excludes that possibility), and the method can also include one or more other steps which are carried out before any of the defined steps, between two of the defined steps, or after all of the defined steps (except where context excludes that possibility).

Thus, the present invention is well adapted to carry out the objectives and attain the ends and advantages mentioned above as well as those inherent therein. While presently preferred embodiments have been described for purposes of this disclosure, numerous changes and modifications will be apparent to those of ordinary skill in the art. Such changes and modifications are encompassed within the spirit of this invention as defined by the claims.

1. A method of eliciting an immune response against at least one Salmonella serovar in a subject in need thereof, comprising
administering to the subject a composition comprising
at least one Salmonella pathogenicity island 1 (SPI-1) and/or Salmonella pathogenicity island 2 (SPI-2) extracellular protein; and
a physiologically acceptable carrier;
wherein said composition is administered in an amount so as to elicit an immune response to said at least one Salmonella serovar in said subject.

2. The method of claim 1, wherein said composition further comprises an adjuvant.

3. The method of claim 1, wherein said composition comprises an extracellular protein selected from the group consisting of: SipD, SipB, SseB and SseC.

4. The method of claim 3, wherein said composition comprises SipD and SipB.

5. The method of claim 4, wherein said composition further comprises SseB.

6. The method of claim 1, wherein said Salmonella serovar is Salmonella enterica serovar.

7. The method of claim 6, wherein said at least one Salmonella enterica serovar is selected from the group consisting of: typhoid serovar Typhi, typhoid serovar Paratyphi A, typhoid serovar Paratyphi B, non-typhoidal serovar Typhimurium and non-typhoidal serovar Enteritidis.

8. The method of claim 1, wherein said subject is selected from a human and an agricultural animal.

9. The method of claim 8, wherein said agricultural animal is selected from the group consisting of: cattle, poultry, swine, horses, sheep and goats.

10. An immunogenic composition, comprising
at least one Salmonella pathogenicity island 1 (SPI-1) and/or Salmonella pathogenicity island 2 (SPI-2) extracellular protein; and
a physiologically acceptable carrier.

11. The immunogenic composition of claim 10, wherein said immunogenic composition further comprises an adjuvant.

12. The immunogenic composition of claim 10, wherein said at least one SPI-1 and/or SPI-2 extracellular protein is selected from the group consisting of:
SipD, SipB, SseB and SseC.

13. The immunogenic composition of claim 12, wherein said at least one SPI-1 and/or SPI-2 extracellular protein includes SipD and SipB.

14. The immunogenic composition of claim 13, further comprising SseB.

15. A method of treating or preventing Salmonella infection by one or both of a typhoid Salmonella serovar and a non-typhoid Salmonella serovar in a subject in need thereof, comprising
administering to the subject an amount of a composition of any one of claims 10-14 sufficient to treat or prevent said Salmonella infection in said subject.

16. A method of lessening the severity of symptoms of Salmonella infection in a subject in need thereof, comprising
administering to the subject an amount of a composition of any one of claims 10-14 sufficient to lessen the severity of said symptoms in said subject.

17. A method of decreasing fecal shedding of Salmonella from a subject who is or is likely to be infected with Salmonella, comprising
administering to the subject an amount of a composition of any one of claims 10-14 sufficient to lessen the severity of said symptoms in said subject.


SEQUENCE LISTING (8 sequences)

SEQ ID NO: 1 - protein, 593 aa, Salmonella enterica
MVNDASSISRSGYTQNPRLAEAAFEGVRKNTDFLKAADKAFKDVVATKAGDLKAGTKSGESAINTVGLKPPTDAAREKLSSEGQLTLLLGKLMTLLGDVSLSQLESRLAVWQAMIESQKEMGIQVSKEFQTALGEAQEATDLYEASIKKTDTAKSVYDAATKKLTQAQNKLQSLDPADPGYAQAEAAVEQAGKEATEAKEALDKATDATVKAGTDAKAKAEKADNILTKFQGTANAASQNQVSQGEQDNLSNVARLTMLMAMFIEIVGKNTEESLQNDLALFNALQEGRQAEMEKKSAEFQEETRKAEETNRIMGCIGKVLGALLTIVSVVAAVFTGGASLALAAVGLAVMVADEIVKAATGVSFIQQALNPIMEHVLKPLMELIGKAITKALEGLGVDKKTAEMAGSIVGAIVAAIAMVAVIVVVAVVGKGAAAKLGNALSKMMGETIKKLVPNVLKQLAQNGSKLFTQGMQRITSGLGNVGSKMGLQTNALSKELVGNTLNKVALGMEVTNTAAQSAGGVAEGVFIKNASEALADFMLARFAMDQIQQWLKQSVEIFGENQKVTAELQKAMSSAVQQNADASRFILRQSRA

SEQ ID NO: 2 - DNA, 1782 bp, Salmonella enterica
ttatgcgcgactctggcgcagaataaaacgcgaagcatccgcattttgctgtaccgcagaagacatggctttttgcagttccgccgttaccttctggttttcaccaaatatttctacggattgtttaagccactgctgaatctgatccatggcaaaacgggcgagcataaaatcagcaagcgcctcgctggcatttttaataaatacgccctcggcaacaccaccggctgactgggctgcggtattcgtgacttccatgcccaacgccactttatttagggtattacctaccagctctttacttaaggcattcgtttgcaggcccatcttgctacccacattacccagaccgctagtaatacgttgcatcccctgggtaaagagtttgctgccgttttgcgccaactgtttcagcacgttaggcaccaacttcttaatcgtttcgcccatcattttgctcagcgcgttacccagtttcgccgccgcgcctttcccgacaactgcgaccaccacaatgaccgccaccatggcaatagcggcgacaatcgcaccaacaatgctgccggccatctctgccgttttcttatcgacgcctaatccttccagcgctttggtaatcgccttgccaatcagctccattaacggcttcagcacatgctccataatcgggtttagcgcctgctgaataaacgacactcccgtcgccgccttcacaatttcatcggccaccattaccgcaagtcccaccgcagccagcgccagactcgccccaccggtaaaaacagcggccacaacgctgacaatggttagcagcgcgccgaggactttcccgatacatcccataatgcggttcgtttcctcggctttgcgcgtctcttcctggaattcagccgatttcttttccatctccgcctgacgcccttcctgcaaggcgttgaaaagcgcaagatcgttttgcaggctttcttccgtatttttgcccacaatctcaataaacatggccatgagcatagtgaggcgggcgacatttgacagattatcctgctcaccctgggaaacctgattctgagaggcggcattagccgttccctggaatttggtcagaatgttatccgctttctcggctttcgctttggcgtctgtgcctgctttaaccgtcgcatccgtggccttatctaaggcctctttcgcctctgtcgcttcttttccggcctgttctaccgcggcttcagcttgtgcatagccggggtcagccgggtccagcgattgcaatttattttgcgcctgcgtcagttttttggtcgcagcgtcataaacactcttggcggtatccgtctttttgatactggcttcatagagatccgtcgcctcctgagcctctcccagagccgtctggaattctttcgatacctgaatccccatctctttttgtgactcaatcatcgcctgccataccgccagacgagactccagttgagacagcgaaacatcgcccagtagggtcattaacttgccaagcagtaatgtcaattgcccttcgctggagagtttttcccgggcggcgtccgtaggcggctttagacccaccgtattaatagcgctctcgccggactttgttccggctttaaggtcgcccgctttcgttgccaccacatctttaaaagctttatccgccgcttttaaaaagtccgtgttcttacgaacgccttcaaaagccgcctcagcgaggcgcggattttgggtatatccgctacggctaatgctacttgcgtcatttaccat

SEQ ID NO: 3 - protein, 339 aa, Salmonella enterica
MLNIQNYSASPHPGIVAERPQTPSASEHAEIAVVPSTTEHRGTDIISLSQAATKIQQAQQTLQSTPPISEENNDERTLARQQLTSSLNALAKSGVSLSAEQNENLRSTFSARRRPYLALRLWPARTTISDAEIWDMVSQNISAIGDSYLGVYENVVAVYTDFYQAFSDILSKMGGWLSPGKDGNTIKLNVDSLKSEISSLINKYTQINKNTILFPSQTGSGMTTATKAEAEQWIKELNLPDSCLKASGSGYVVLVDTGPLSKMVSDLNGIGSGSALELDNAKYQAWQSGFKAQEENLKTTLQTLTQKYSNANSLYDNLVKVLSSTISSSLETAKSFLQG

SEQ ID NO: 4 - DNA, 1020 bp, Salmonella enterica
atgcttaatattcaaaattattccgcttctcctcatccggggatcgttgccgaacggccgcagactccttcggcgagcgagcacgccgagattgccgtggtaccgtctaccacagaacatcgcggcacagatatcatttcattatcgcaggcggctactaaaatccagcaggcacagcagacgctgcagtcaacgccaccgatttctgaagagaataatgacgagcgcacgctggcgcgccaacagttgaccagcagcctgaatgcgctggcgaagtccggcgtgtcattatccgcagaacaaaatgagaacctgcggagcacgttttctgcgcgacgtcggccttatttagcgcttcgcctatggccagcgagaacaaccatttctgatgctgagatttgggatatggtttcccaaaatatatcggcgataggtgacagctacctgggcgtttatgaaaacgttgtcgcagtctataccgatttttatcaggccttcagtgatattctttccaaaatgggaggctggttatcgcctggtaaggatggaaataccattaagctaaatgttgactcacttaaaagtgaaataagtagtttaattaataaatacactcaaataaataaaaatacgattttatttccctcgcaaactggcagcggaatgacaacagcaacgaaagcggaagctgagcagtggattaaagaattgaatttaccggacagctgtctaaaggcgtctggttctggttatgtcgtactggtggatacggggccactgagcaaaatggttagcgatcttaatggaataggatcgggttcagcccttgaactggataacgccaaatatcaagcctggcagtcgggttttaaagcacaggaagaaaatctgaaaaccacattacagacgctgacgcaaaaatatagcaatgccaattcattgtacgacaacctggtaaaagtgctgagcagtacgataagtagcagcctggaaaccgccaaaagcttcctgcaaggataa

SEQ ID NO: 5 - protein, 196 aa, Salmonella enterica
MSSGNILWGSQNPIVFKNSFGVSNADTGSQDDLSQQNPFAEGYGVLLILLMVIQAIANNKFIEVQKNAERARNTQEKSNEMDEVIAKAAKGDAKTKEEVPEDVIKYMRDNGILIDGMTIDDYMAKYGDHGKLDKGGLQAIKAALDNDANRNTDLMSQGQITIQKMSQELNAVLTQLTGLISKWGEISSMIAQKTYS

SEQ ID NO: 6 - DNA, 591 bp, Salmonella enterica
atgtcttcaggaaacatcttatggggaagtcaaaaccctattgtgtttaaaaatagcttcggcgtcagcaacgctgataccgggagccaggatgacttatcccagcaaaatccgtttgccgaagggtatggtgttttgcttattctccttatggttattcaggctatcgcaaataataaatttattgaagtccagaagaacgctgaacgtgccagaaatacccaggaaaagtcaaatgagatggatgaggtgattgctaaagcagccaaaggggatgctaaaaccaaagaggaggtgcctgaggatgtaattaaatacatgcgtgataatggtattctcatcgatggtatgaccattgatgattatatggctaaatatggcgatcatgggaagctggataaaggtggcctacaggcgatcaaagcggctttggataatgacgccaaccggaataccgatcttatgagtcaggggcagataacaattcaaaaaatgtctcaggagcttaacgctgtccttacccaactgacagggcttatcagtaagtggggggaaatttccagtatgatagcgcagaaaacgtactcatga

SEQ ID NO: 7 - protein, 934 aa, Artificial Sequence
  Synthetic SipB-SipD chimera
MLNIQNYSASPHPGIVAERPQTPSASEHAEIAVVPSTTEHRGTDIISLSQAATKIQQAQQTLQSTPPISEENNDERTLARQQLTSSLNALAKSGVSLSAEQNENLRSTFSARRRPYLALRLWPARTTISDAEIWDMVSQNISAIGDSYLGVYENVVAVYTDFYQAFSDILSKMGGWLSPGKDGNTIKLNVDSLKSEISSLINKYTQINKNTILFPSQTGSGMTTATKAEAEQWIKELNLPDSCLKASGSGYVVLVDTGPLSKMVSDLNGIGSGSALELDNAKYQAWQSGFKAQEENLKTTLQTLTQKYSNANSLYDNLVKVLSSTISSSLETAKSFLQGVDMVNDASSISRSGYTQNPRLAEAAFEGVRKNTDFLKAADKAFKDVVATKAGDLKAGTKSGESAINTVGLKPPTDAAREKLSSEGQLTLLLGKLMTLLGDVSLSQLESRLAVWQAMIESQKEMGIQVSKEFQTALGEAQEATDLYEASIKKTDTAKSVYDAATKKLTQAQNKLQSLDPADPGYAQAEAAVEQAGKEATEAKEALDKATDATVKAGTDAKAKAEKADNILTKFQGTANAASQNQVSQGEQDNLSNVARLTMLMAMFIEIVGKNTEESLQNDLALFNALQEGRQAEMEKKSAEFQEETRKAEETNRIMGCIGKVLGALLTIVSVVAAVFTGGASLALAAVGLAVMVADEIVKAATGVSFIQQALNPIMEHVLKPLMELIGKAITKALEGLGVDKKTAEMAGSIVGAIVAAIAMVAVIVVVAVVGKGAAAKLGNALSKMMGETIKKLVPNVLKQLAQNGSKLFTQGMQRITSGLGNVGSKMGLQTNALSKELVGNTLNKVALGMEVTNTAAQSAGGVAEGVFIKNASEALADFMLARFAMDQIQQWLKQSVEIFGENQKVTAELQKAMSSAVQQNADASRFILRQSRA

SEQ ID NO: 8 - DNA, 2805 bp, Artificial Sequence
  Synthetic nucleic acid sequence encoding SipB-SipD chimera
atgcttaatattcaaaattattccgcttctcctcatccggggatcgttgccgaacggccgcagactccttcggcgagcgagcacgccgagattgccgtggtaccgtctaccacagaacatcgcggcacagatatcatttcattatcgcaggcggctactaaaatccagcaggcacagcagacgctgcagtcaacgccaccgatttctgaagagaataatgacgagcgcacgctggcgcgccaacagttgaccagcagcctgaatgcgctggcgaagtccggcgtgtcattatccgcagaacaaaatgagaacctgcggagcacgttttctgcgcgacgtcggccttatttagcgcttcgcctatggccagcgagaacaaccatttctgatgctgagatttgggatatggtttcccaaaatatatcggcgataggtgacagctacctgggcgtttatgaaaacgttgtcgcagtctataccgatttttatcaggccttcagtgatattctttccaaaatgggaggctggttatcgcctggtaaggatggaaataccattaagctaaatgttgactcacttaaaagtgaaataagtagtttaattaataaatacactcaaataaataaaaatacgattttatttccctcgcaaactggcagcggaatgacaacagcaacgaaagcggaagctgagcagtggattaaagaattgaatttaccggacagctgtctaaaggcgtctggttctggttatgtcgtactggtggatacggggccactgagcaaaatggttagcgatcttaatggaataggatcgggttcagcccttgaactggataacgccaaatatcaagcctggcagtcgggttttaaagcacaggaagaaaatctgaaaaccacattacagacgctgacgcaaaaatatagcaatgccaattcattgtacgacaacctggtaaaagtgctgagcagtacgataagtagcagcctggaaaccgccaaaagcttcctgcaaggagtcgacttatgcgcgactctggcgcagaataaaacgcgaagcatccgcattttgctgtaccgcagaagacatggctttttgcagttccgccgttaccttctggttttcaccaaatatttctacggattgtttaagccactgctgaatctgatccatggcaaaacgggcgagcataaaatcagcaagcgcctcgctggcatttttaataaatacgccctcggcaacaccaccggctgactgggctgcggtattcgtgacttccatgcccaacgccactttatttagggtattacctaccagctctttacttaaggcattcgtttgcaggcccatcttgctacccacattacccagaccgctagtaatacgttgcatcccctgggtaaagagtttgctgccgttttgcgccaactgtttcagcacgttaggcaccaacttcttaatcgtttcgcccatcattttgctcagcgcgttacccagtttcgccgccgcgcctttcccgacaactgcgaccaccacaatgaccgccaccatggcaatagcggcgacaatcgcaccaacaatgctgccggccatctctgccgttttcttatcgacgcctaatccttccagcgctttggtaatcgccttgccaatcagctccattaacggcttcagcacatgctccataatcgggtttagcgcctgctgaataaacgacactcccgtcgccgccttcacaatttcatcggccaccattaccgcaagtcccaccgcagccagcgccagactcgccccaccggtaaaaacagcggccacaacgctgacaatggttagcagcgcgccgaggactttcccgatacatcccataatgcggttcgtttcctcggctttgcgcgtctcttcctggaattcagccgatttcttttccatctccgcctgacgcccttcctgcaaggcgttgaaaagcgcaagatcgttttgcaggctttcttccgtatttttgcccacaatctcaataaacatggccatgagcatagtgaggcgggcgacatttgacagattatcctgctcaccctgggaaacctgattctgagaggcggcattagccgttccctggaatttggtcagaatgttatccgctttctcggctttcgctttggcgtctgtgcctgctttaaccgtcgcatccgtggccttatctaaggcctctttcgcctctgtcgcttcttttccggcctgttctaccgcggcttcagcttgtgcatagccggggtcagccgggtccagcgattgcaatttattttgcgcctgcgtcagttttttggtcgcagcgtcataaacactcttggcggtatccgtctttttgatactggcttcatagagatccgtcgcctcctgagcctctcccagagccgtctggaattctttcgatacctgaatccccatctctttttgtgactcaatcatcgcctgccataccgccagacgagactccagttgagacagcgaaacatcgcccagtagggtcattaacttgccaagcagtaatgtcaattgcccttcgctggagagtttttcccgggcggcgtccgtaggcggctttagacccaccgtattaatagcgctctcgccggactttgttccggctttaaggtcgcccgctttcgttgccaccacatctttaaaagctttatccgccgcttttaaaaagtccgtgttcttacgaacgccttcaaaagccgcctcagcgaggcgcggattttgggtatatccgctacggctaatgctacttgcgtcatttaccat